(19) United States
(12) Patent Application Publication
(10) Pub. No.: US 2007/0224299 A1
(43) Pub. Date: Sep. 27, 2007

(54) WEIGHT LOSS WITH CITRUS FLAVONOIDS

(76) Inventor: Shawn M. Talbot, Draper, UT (US)

Correspondence Address: HOLME ROBERTS & OWEN, LLP, 299 SOUTH MAIN, SUITE 1800, SALT LAKE CITY, UT, 84111, US

(21) Appl. No.: 11/690,714

(22) Filed: Mar. 23, 2007

Related U.S. Application Data
(60) Provisional application No. 60/785,158, filed on Mar. 23, 2006.

(51) Int. Cl. A61K 36/752; A61K 31/7048; A61K 31/353

(52) U.S. Cl. 424/736; 514/27; 514/456

(57) ABSTRACT

A therapeutically effective dose of polymethoxylated flavones, for example in the form of citrus peel extract, is provided for reducing the typical rise in cortisol levels in a mammal undergoing weight loss. The polymethoxylated flavones may include one or more of tangeretin, nobiletin, and sinensetin. The polymethoxylated flavones may be administered in any of a variety of known modes of administrations such as oral and topical.

### CROSS-REFERENCE TO RELATED APPLICATIONS

This application claims the benefit of Provisional Application No. 60/785,158, filed Mar. 23, 2006, entitled “Weight Loss With Citrus Flavonoids,” which is incorporated by reference herein in its entirety.

### BACKGROUND OF THE INVENTION

1. The Field of the Invention

The present invention relates to the fields of food compositions and dietary supplements. More particularly, the invention provides compositions and methods for promoting weight reduction in a human subject by administering an effective dose of citrus peel extract to a human subject undergoing weight loss to reduce cortisol levels.

2. The Relevant Technology

Obesity, diabetes, and metabolic syndrome (“Syndrome X”—encompassing insulin resistance, type 2 diabetes, dyslipidemia, hypertension, and abdominal obesity) are reaching epidemic proportions in the United States and around the world. Excess glucocorticoids such as cortisol (a primary stress hormone) have been theorized to contribute high levels of abdominal fat and diabetes. The negative effects of cortisol have been theorized in part due to an increased appetite that has been reported in persons with elevated cortisol levels. Further, high levels of urinary cortisol excretion has been noted in persons exercising high dietary restraint. The high levels of cortisol excretion suggest a harmful cycle in which attempts to lose weight by eating less are countered by the body releasing cortisol in response to the stress, which in turn increases appetite and thus heightens the chances of a failed weight loss attempt.

Accordingly, methods of reducing the harmful affects of cortisol would represent an advance in the art of dietary supplements

### BRIEF SUMMARY OF THE INVENTION

According the invention, conditions enhanced by the presence of excess glucocorticoids such as cortisol can be reduced by using citrus peel extract, including polymethoxylated flavones (PMFs), to reduce the increase in cortisol levels typically resulting from body fat and with weight loss. The citrus peel extractor PMFs may be administered in any of a variety of known modes of administrations such as oral and topical.

Accordingly, a first example embodiment of the invention is a composition of matter including an effective dose of citrus peel extract for inhibiting weight gain or promoting weight loss in a mammal. An effective dose of the citrus peel extract may be from about 1 mg to about 1000 mg, for example, or more preferably the effective dose may be from about 100 mg to about 500 mg of a citrus peel extract. Regardless, the citrus peel extract comprises at least about 10 mg PMFs in the citrus peel extract. The PMFs preferably include at least one of tangeretin, nobiletin, and sinensetin.

Example compositions of the present invention may be formulated for administration through various known routes of administration, such as oral, parenteral, and transdermal routes. Examples of oral dosage formulations include without limitation, tablets, capsules, liquids, suspensions, gels, powders, effervescent beverages, lozenges, chewing gum, candy, supplemented food, etc. Examples of transdermal routes of administration include without limitation, topical formulations, such as lotions, creams, gels, and pastes, and transdermal patches, such as liquid reservoir patches, plasters, and adhesive matrix patches. Suitable ingredients required to produce a particular formulation, such as specific carriers, excipients, binders, penetration enhancers, etc., will be readily recognized by those of ordinary skill in the art and are not discussed in detail herein to avoid obscuring the invention. In preferred embodiments one or more weight loss enhancing additives such as ginseng, caffeine, guarana, green tea extract, chromium, vanadium, and CLA are added.

Another example embodiment of the invention is a method for promoting weight loss in a mammal. The method generally includes: administering to a mammal participating in a weight loss exercise routine a diet consisting of a caloric intake that is insufficient to maintain the mammal's weight; and administering to the mammal a an effective dose of citrus peel extract to either reduce cortisol levels in the mammal or reduce the amount of cortisol increase in the mammal.

In another example embodiment of the invention the citrus peel extract is administered to the human subject as part of a regular regime of dose administrations and the timing and the amount of each dosage is determined with respect the cumulative effects of the regime. In this example the effective dose may optionally be defined as the amount sufficient to reduce cortisol levels by at least 5% during the regime relative to a cortisol level that would result in the absence of the administration of the citrus peel extract.

These and other objects and features of the present invention will become more fully apparent from the following description and appended claims, or may be learned by the practice of the invention as set forth hereinafter.

### DETAILED DESCRIPTION OF THE PREFERRED EMBODIMENTS

In the following description, numerous specific details are set forth in order to provide a thorough understanding of the present invention. It will be obvious, however, to one skilled in the art that the present invention may be practiced without these specific details. In other instances, well-known aspects of citrus peel extract, polymethoxylated flavones (PMFs), and various oral and topical dosage forms have not been described in particular detail in order to avoid unnecessarily obscuring the present invention.

Polymethoxylated Flavones (PMFs), particularly tangeretin, sinensetin, and nobilitin, have known health benefits in reducing cholesterol and promoting cardiovascular health. According to the invention it has been determined that PMFs also assist in weight loss or the reduction in weight gain (weight loss and the reduction in weight gain will sometimes be referred to collectively as “weight loss” herein for simplicity). Although all embodiments of the application are not necessarily tied to any particular theory, it is believed that the PMFs reduce cortisol levels, or at least decrease cortisol level gains, during periods of stress to facilitate the weight loss. Since cortisol is a stress hormone, higher levels of which have been tied to weight gain, the reduction in cortisol leads to the weight loss.

Accordingly, the present invention provides compositions and methods using PMFs for the prevention and treatment of diabetes and obesity. Individuals at high risk of developing or having diabetes or desiring to prevent weight gain or promote weight loss may be treated with an effective dose of citrus polymethoxylated flavones (PMFs). More particularly, the present invention relates to the discovery and use of PMFs to reduce systemic and local cortisol concentrations (liver and adipose tissue), and promote blood sugar control and weight loss. The current invention therefore represents an important and innovative treatment for obesity, diabetes, and related metabolic diseases by promoting weight loss and controlling blood sugar levels.

According to one embodiment of the invention, a mammalian subject can be administered once a day a composition of matter having from about 1 mg to about 1,000 mg of a citrus peel extract. In a more preferred embodiment the composition of matter has from about 100 mg to about 500 mg of a citrus peel extract. In a still more preferred embodiment the composition of matter at least about 300 mg of the citrus peel extract. In each of the foregoing examples, the citrus peel extract preferably has at least about 10 mg PMFs, more preferably at least about 80 mg PMFs. The amount of citrus peel extract may increase or decrease depending on the concentration of the citrus peel extract.

Alternatively, in another embodiment of the invention a mammalian subject can be administered once a day a composition of matter having one or more of tangeretin, nobiletin, and sinensetin. By way of example only, the dosage may have one or more of from about 1 mg to about 50 mg of tangeretin, from about 1 mg to about 50 mg of nobiletin, and from about 1 mg to about 20 mg of sinensetin. The levels of each in a particular dosage may increase or decrease depending on the respective concentrations in the citrus peel extract as well as the overall concentration of the citrus peel extract. In a more preferred embodiment the composition of matter has from about 5 mg to about 20 mg of tangeretin, from about 5 mg to about 20 mg of nobiletin, and from about 2 mg to about 10 mg of sinensetin.

One embodiment of the invention is a composition of matter having an effective dose of citrus peel extract for preventing weight gain or promoting weight loss in a mammal. As used herein the term “an effective dose” indicates an amount sufficient to create a desirable result. For example, in one embodiment an effective dose is the amount necessary to obtain a measurable change in cortisol levels. An effective dose will therefore preferably reduce cortisol levels by at least about 5 percent as compared to a cortisol level that would result in the absence of the administered dose(s). Still more preferably, an effective dose will reduce cortisol levels by at least about 10 percent as compared to a cortisol level that would result in the absence of the administered dose(s). Alternatively, an effective dose can be the amount necessary to promote a relative weight reduction, wherein a relative weight loss is defined by a human obtaining a lower weight than the human user would have obtained without receiving the effective dosage. The precise parameters of an effective dose may of course vary with a number of factors, including by way of example only, weight, gender, age, and desired result.

The compositions of the present invention may be formulated for administration through various known routes of administration, such as oral, parenteral, and transdermal routes. Examples of oral dosage formulations include without limitation, tablets, capsules, liquids, suspensions, gels, powders, effervescent beverages, lozenges, chewing gum, candy, supplemented food, etc. Examples of transdermal routes of administration include without limitation, topical formulations, such as lotions, creams, gels, and pastes, and transdermal patches, such as liquid reservoir patches, plasters, and adhesive matrix patches. Suitable ingredients required to produce a particular formulation, such as specific carriers, excipients, binders, penetration enhancers, etc., will be readily recognized by those of ordinary skill in the art and are not discussed in detail herein to avoid obscuring the invention.

For example, one preferred administration route is as a diet shake. The diet shake can be provided to a user in either liquid form or as a powder having suitable ingredients for the user to form a shake upon mixture with a liquid. In addition, a supplemented food such as a nutritional bar or dessert is also preferred.

The citrus peel extract can be preferably combined in the foregoing administration forms with a variety of other supplements, vitamins, stabilizers, and the like as are known in the art to create a desirable administration form. By way of example only, such additives can include other forms of ginseng, caffeine, guarana, green tea extract, chromium, vanadium, CLA, and the like.

Where the dosage form is oral, a flavoring such as a citrus flavoring can be added to improve the taste as well as to enhance the user's perception of consuming the citrus peel extract. Where the dosage form is topical, a scent such as a citrus aroma can be added to not only improve the scent of the topical cream or ointment, but also to enhance the user's awareness of using the citrus peel extract.

Other conventional additives such as stabilizers, pH adjusters, excipients, carriers and diluents and the like may be added as desired or necessary as is known in the art provided they do not interfere with the activity of citrus peel extract.

The compositions of the invention can be formulated so as to provide quick, sustained or delayed release of the citrus peel extract after administration employing procedures and formulations known in the art.

The following example is shown by way of illustration only.

### EXAMPLES

### Example 1

A group of fifty moderately overweight human subjects, self-described as “stress-eaters,” participated in an eight week study. The subjects had an average age of 44, an average starting weight of 78.5 kg, an average starting body fat percent of 32.4%, and an average starting waist circumference of 86.7 cm. During the eight week study, the subjects followed a program that included: daily supplements including citrus peel extract, a moderate calorie-restricted diet based on resting metabolic rate (RMR), a moderate exercise program on five days each week, and daily stress management techniques. More particularly, polymethoxylated Flavones (PMFs) were administered to the test subjects in the form of 300 mg of citrus peel extract/day containing 81 mg of PMFs comprising a minimum of 12 mg tangeretin, 12 mg nobiletin, and 5 mg sinensetin.

At the conclusion of the study the human subjects were again tested and found to have a mean reduced body weight by 0.6 kg, mean reduced body fat by 5.0%, mean reduced fat mass by 1.9 kg, mean increased lean mass by 1.3 kg, and mean reduced waist circumference by 1.3 cm.

In addition, the subjects were tested before and after the eight week study for salivary cortisol levels by salivary enzyme immunoassay. A mean 18.7% reduction in salivary cortisol levels for the subjects was observed suggesting a reduction in stress.

In addition, before and after participation the subjects each completed a 65 question Profile of Mood States (POMS) questionnaire. The Profile of Mood States (POMS) is a well known test that measures present mood state (disturbance) by a list of adjectives. In the test, respondents rate 65 adjectives on a 5-point intensity scale in terms of how they have been feeling in the past week (O=not at all and 4=extremely). Except for vigor, the higher the score, the greater the mood disturbance/more distress. For analysis, the questions of the POMS are divided into six categories: tension, fatigue, confusion, vigor, depression, and anger and a score calculated for each. A Global Mood State score is also derived from the sum of all negative categories minus the score for vigor. For further details on the POMS test, see McNair D M, Lorr M, and Droppleman L F., Profile of Mood States Manual, San Diego, Calif.: Educational and Industrial Testing Services (1971). As noted in Table 1 below, the respondents had mean improvements of from 14% to 41% in all categories suggesting a strong reduction in stress as a result of the study.

TABLE 1

| Test | Percent Change |
|---|---|
| Global Mood State | −19.7% |
| Tension | −19% |
| Fatigue | −41% |
| Confusion | −14% |
| Vigor | +29% |
| Depression | −40% |
| Anger | −41% |

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A composition of matter, comprising:
an effective dose of citrus peel extract for inhibiting weight gain or promoting weight loss in a mammal.

2. The composition of matter of claim 1, wherein the citrus peel extract comprises from about 100 mg to about 500 mg of a citrus peel extract.

3. The composition of matter of claim 1, wherein the citrus peel extract comprises at least about 10 mg polymethoxylated flavones (PMFs).

4. A composition of matter as defined in claim 1, wherein the citrus peel extract comprises at least about 80 mg PMFs.

5. A composition of matter as defined in claim 1, wherein the citrus peel extract comprises at least one of tangeretin, nobiletin, and sinensetin.

6. A composition of matter as defined in claim 1, wherein the citrus peel extract comprises:
from about 5 mg to about 20 mg tangeretin;
from about 5 mg to about 20 mg nobiletin, and
from about 2 mg to about 10 mg sinensetin.

7. A topical supplement, comprising:
the composition of claim 1; and
a topical cream blended with the composition of claim 1, the topical cream being configured for facilitating transdermal absorption of the citrus peel extract.

8. A dietary supplement, comprising:
a dietary acceptable carrier prepared in a dosage form selected from the group consisting of a liquid, a tablet, a capsule, a powder, a confectionary, and a supplemented food; and
a composition as defined in claim 1.

9. The dietary supplement of claim 8, further comprising one or more additives selected from the group consisting of: ginseng, caffeine, guarana, green tea extract, chromium, vanadium, and CLA.

10. A method for promoting weight loss in a mammal, the method comprising:
administering to a mammal participating in a weight loss exercise routine a diet consisting of a caloric intake that is insufficient to maintain the mammal's weight; and
administering to the mammal a composition of matter as defined as in claim 1, the citrus peel extract comprising a sufficient amount to either reduce cortisol levels in the mammal or reduce the amount of cortisol increase in the mammal.

11. A method for promoting weight reduction in a mammal, the method comprising administering to a mammal undergoing loss of body fat an effective dose of citrus peel extract for reducing cortisol levels or inhibiting cortisol level increases in the mammal.

12. The method of claim 11, wherein the mammal comprises a human.

13. The method of claim 11, further comprising administering to the mammal the effective dose at least once daily for a plurality of days.

14. The method of claim 11, wherein the citrus peel extract comprising at least one of tangeretin, nobiletin, and sinensetin.

15. The method of claim 11, wherein the citrus peel extract is administered to the human subject as part of a regular regime of dose administrations and the timing and the amount of each dosage is determined with respect the cumulative effects of the regime.

16. The method of claim 15, wherein the effective dose is defined as sufficient to reduce cortisol levels by at least 5% during the regime relative to a cortisol level that would result in the absence of the administration of the citrus peel extract.

17. The method of claim 11, wherein the citrus peel extract is in a oral form selected from the group consisting of a beverage, a tablet, a capsule, a powder, a confectionary, and a supplemented food.

18. The method of claim 11, wherein the citrus peel extract is blended with a topical cream for topical administration and administering the effective dose of citrus peel extract comprises topically administering the citrus peel extract.

19. A method for preventing or treating conditions enhanced by the presence of excess glucocorticoids in a mammal, the method comprising:
administering to a mammal in need thereof a therapeutically effective dose of citrus peel extract for reducing excess glucocorticoids, the citrus peel extract comprising at least about 10 mg of polymethoxylated flavones (PMFs).

20. The method of claim 19, wherein the therapeutically effective dose of citrus peel extract comprises from about 100 mg to about 500 mg.
